(19) United States
(12) Patent Application Publication
(10) Pub. No.: US 2022/0265164 A1
(43) Pub. Date: Aug. 25, 2022

(54) METHOD AND SYSTEM OF CAPNOGRAPHY

(71) Applicant: INCOBA, LLC, Chesterfield, MO (US)

(72) Inventor: Alonzo C. AYLSWORTH, Wildwood, MO (US)

(73) Assignee: INCOBA, LLC, Chesterfield, MO (US)

(21) Appl. No.: 17/743,128

(22) Filed: May 12, 2022

Related U.S. Application Data
(63) Continuation of application No. PCT/US2020/060135, filed on Nov. 12, 2020.
(60) Provisional application No. 62/936,743, filed on Nov. 18, 2019.

(51) Int. Cl. A61B 5/083; A61B 5/097; A61M 16/06; A61M 16/00; G16H 40/63; G16H 20/40

(52) U.S. Cl. CPC A61B 5/0836; A61B 5/097; A61M 16/0666; A61M 16/024; G16H 40/63; G16H 20/40; A61M 2230/432

(57) ABSTRACT

At least one example embodiment is a method of generating a capnographic waveform, the method including: measuring carbon dioxide in exhaled gas flowing in a first flow path, the measuring creates a first set of values indicative of carbon dioxide; measuring, by the controller of the device, carbon dioxide in exhaled gas flowing in a second flow path distinct from the first flow path, the measuring creates a second set of values indicative of carbon dioxide; and creating, by the controller of the device, a capnographic waveform. Creating the capnographic waveform may including using the first set of values indicative of carbon dioxide, the second set of values indicative of carbon dioxide, and/or both the first and second sets of values of carbon dioxide.

### CROSS-REFERENCE TO RELATED APPLICATIONS

This application is a continuation of PCT App. No. PCT/US2020/060135 filed Nov. 12, 2020. The PCT application claims the benefit of U.S. Provisional Application No. 62/936,743 filed Nov. 18, 2019 and titled “Method and System of Capnography.” Both the PCT application and the provisional application are incorporated by reference herein as if reproduced in full below.

### BACKGROUND

Capnography refers to monitoring of carbon dioxide in an exhalation. If carbon dioxide readings during an exhalation are plotted, the shape or envelope of a single exhalation may provide valuable information regarding state of the patient's respiration. Relatedly, if a series of readings of carbon dioxide are plotted, the series of readings may also provide value information regarding the state of the patient's respiration, such hypoventilation and hyperventilation.

Any method or system that improves capnography would provide better patient outcomes.

### SUMMARY

One example embodiment is a method of generating a capnographic waveform, the method comprising: measuring, by a controller of a device, carbon dioxide in exhaled gas flowing in a first flow path, the measuring creates a first set of values indicative of carbon dioxide; measuring, by the controller of the device, carbon dioxide in exhaled gas flowing in a second flow path distinct from the first flow path, the measuring creates a second set of values indicative of carbon dioxide; and creating, by the controller of the device, a capnographic waveform. Creating the capnographic waveform may use at least one selected from the group comprising—the first set of values indicative of carbon dioxide, the second set of values indicative of carbon dioxide, and/or both the first and second sets of values of carbon dioxide.

In the example method: measuring the exhaled gas flow in the first flow path may further comprise measuring exhaled gas flow from a first lumen of a nasal cannula; and measuring the exhaled gas flow in the second flow path may further comprise measuring exhaled gas flow from a second lumen of the nasal cannula, the first lumen fluidly isolated from the second lumen.

In the example method: measuring exhaled gas flow in the first flow path may further comprises measuring exhaled gas flow from a first lumen of a bifurcated nasal cannula; and measuring exhaled gas flow in the second flow path may further comprise measuring exhaled gas flow from a second lumen of the bifurcated nasal cannula.

In the example method, creating the capnographic waveform may further comprise combining the first and second sets of values indicative of carbon dioxide. Combining the first and second sets of values of carbon dioxide may further comprise averaging corresponding values in the first and second sets of values indicative of carbon dioxide. Averaging corresponding values may further comprise averaging the values of the first and second sets of values indicative of carbon dioxide at corresponding points in time. Averaging corresponding values may further comprise averaging the values of the first and second sets of values indicative of carbon dioxide at corresponding points in accumulated volume of exhaled gas.

The example method may further comprise: sensing an inhalation; and providing a volume of therapeutic gas triggered by the sensing and based on the capnographic waveform.

Another example embodiment is a first system comprising: a controller; a first carbon dioxide sensor (CO2 sensor) fluidly coupled to a first hose connection, and communicatively coupled to the controller; and a second CO2 sensor fluidly coupled to a second hose connection, and communicatively coupled to the controller. The controller may be configured to: read a first set of values indicative of carbon dioxide from the first CO2 sensor; read a second set of values indicative of carbon dioxide from the second CO2 sensor; and create a capnographic waveform using at least one of the first and second sets of values indicative of carbon dioxide.

The example first system may further comprise: a first sensor electrically coupled to the controller and configured to fluidly couple to the first hose connection, the first sensor senses an attribute of airflow through the first hose connection; and a second sensor electrically coupled to the controller and configured to fluidly couple to the second hose connection, the second sensor senses an attribute of airflow of the second hose connection. When the controller creates the capnographic waveform, the controller may be further configured to: read the attribute of airflow from the first sensor; read the attribute of airflow from the second sensor; utilize the first set of values indicative of carbon dioxide if the attribute of airflow from the first sensor is above a predetermined threshold; and utilize the second set of values indicative of carbon dioxide if the attribute of airflow from the second sensor is above a predetermined threshold.

In the example first system, when the controller creates the capnographic waveform, the controller may create the capnographic waveform using both the first and second sets of values indicative of carbon dioxide. When the controller creates the capnographic waveform, the controller may be configured to average corresponding values in the first and second sets of values indicative of carbon dioxide. When the controller averages, the controller may be further configured to average the values of the first and second sets of values indicative of carbon dioxide at corresponding points in time. When the controller averages, the controller may be further configured to average the values of the first and second sets of values indicative of carbon dioxide at corresponding points in accumulated volume.

The example first system may further comprise: a first sensor electrically coupled to the controller and configured to fluidly couple to the first hose connection, the first sensor senses an attribute of airflow through the first hose connection; a first valve electrically coupled to the controller and configured to fluidly couple a source hose connection to the first hose connection; a second sensor electrically coupled to the controller and configured to fluidly couple to the second hose connection, the second sensor senses an attribute of airflow of the second hose connection; a second valve electrically coupled to the controller and configured to fluidly couple the source hose connection to the second hose connection. The controller may be further configured to: sense an inhalation by way of the first sensor or the second sensor; provide, based on the sensing, a flow of therapeutic gas to the first hose connection by way of the first valve. The controller may be further configured to modify a volume of therapeutic gas provided in a subsequent inhalation based on the capnographic waveform.

Another example embodiment is a second system comprising: a source of therapeutic gas; a bifurcated nasal cannula; a therapeutic gas delivery device coupled to the source of therapeutic gas, and coupled to the bifurcated nasal cannula. The therapeutic gas delivery device may comprise: a controller; a first carbon dioxide sensor (CO2 sensor) fluidly coupled to a first lumen of the bifurcated nasal cannula, and communicatively coupled to the controller; a second CO2 sensor fluidly coupled to a second lumen of the bifurcated nasal cannula, and communicatively coupled to the controller; a first sensor electrically coupled to the controller and configured to fluidly couple to the first lumen of the bifurcated nasal cannula, the first sensor senses an attribute of airflow through the first lumen; a first valve electrically coupled to the controller and configured to fluidly couple the source of therapeutic gas to the first lumen; a second sensor electrically coupled to the controller and configured to fluidly couple to the second lumen of the bifurcated nasal cannula, the second sensor senses an attribute of airflow of the second lumen; a second valve electrically coupled to the controller and configured to fluidly couple the source of therapeutic gas to the second lumen. The controller may be configured to: sense an inhalation by way of the first sensor or the second sensor; provide a flow of therapeutic gas to the first lumen of the bifurcated nasal cannula; read a first set of values indicative of carbon dioxide from the first CO2 sensor during an exhalation; read a second set of values indicative of carbon dioxide from the second CO2 sensor during the exhalation; and create a capnographic waveform using at least one of the first and second sets indicative of carbon dioxide.

In the example second system, when the controller creates the capnographic waveform, the controller may be further configured to: read the attribute of airflow from the first sensor; read the attribute of airflow from the second sensor; utilize the first set of values indicative of carbon dioxide if the attribute of airflow from the first sensor is above a predetermined threshold; and utilize the second set of values indicative of carbon dioxide if the attribute of airflow from the second sensor is above a predetermined threshold. When the controller creates the capnographic waveform, the controller may be further configured to average corresponding values in the first and second sets of values indicative of carbon dioxide.

In the example second system, the controller may be further configured to control a volume of therapeutic gas delivered based on a capnographic waveform associated with a prior exhalation.

### BRIEF DESCRIPTION OF THE DRAWINGS

For a detailed description of example embodiments, reference will now be made to the accompanying drawings in which:

FIG. 1 shows a plot of a capnographic waveform as a function of time;

FIG. 2 shows a plot of a capnographic waveform as a function of time;

FIG. 3 shows a series of capnographic waveforms as a function of time;

FIG. 4 shows a system in accordance with at least some embodiments;

FIG. 5 shows a system in accordance with at least some embodiments;

FIG. 6 show a method in accordance with at least some embodiments; and

FIG. 7 shows a controller in accordance with at least some embodiments.

### DEFINITIONS

Various terms are used to refer to particular system components. Different companies may refer to a component by different names— this document does not intend to distinguish between components that differ in name but not function. In the following discussion and in the claims, the terms “including” and “comprising” are used in an open-ended fashion, and thus should be interpreted to mean “including, but not limited to . . . ” Also, the term “couple” or “couples” is intended to mean either an indirect or direct connection. Thus, if a first device couples to a second device, that connection may be through a direct connection or through an indirect connection via other devices and connections.

“Capnographic waveform” shall mean a series of values indicative of carbon dioxide in an exhalation, the series of values as a function of another parameter (e.g., time of exhalation, or volume of exhalation). “Capnographic waveform” shall not require a plot of the waveform.

“Carbon dioxide percentage” shall mean any measure of the relationship of carbon dioxide in exhaled gas.

“Nares” shall mean the nostrils of a patient.

“Naris” shall mean a single nostril of a patient.

“Substantially”, in relation to a recited volume, shall mean within +/−10% of the recited volume.

In relation to electrical devices (whether stand alone or as part of an integrated circuit), the terms “input” and “output” refer to electrical connections to the electrical devices, and shall not be read as verbs requiring action. For example, a controller may have sense input coupled to a sensor. The example “sense input” defines an electrical connection to the controller, and shall not be read to require inputting signals to the controller.

“Assert” shall mean changing the state of a Boolean signal. Boolean signals may be asserted high or with a higher voltage, and Boolean signals may be asserted low or with a lower voltage, at the discretion of the circuit designer. Similarly, “de-assert” shall mean changing the state of the Boolean signal to a voltage level opposite the asserted state.

“Controller” shall mean, alone or in combination, individual circuit components, an application specific integrated circuit (ASIC), a field programmable gate array (FPGA), a microcontroller with controlling software, a digital signal processor (DSP), a processor with controlling software, a programmable logic device (PLD), or a computer system with controlling software configured to read inputs and drive outputs responsive to the inputs.

### DETAILED DESCRIPTION

The following discussion is directed to various embodiments of the invention. Although one or more of these embodiments may be preferred, the embodiments disclosed should not be interpreted, or otherwise used, as limiting the scope of the disclosure, including the claims. In addition, one skilled in the art will understand that the following description has broad application, and the discussion of any embodiment is meant only to be exemplary of that embodiment, and not intended to intimate that the scope of the disclosure, including the claims, is limited to that embodiment.

Before delving into the various example embodiments, the specification starts with a discussion of why capnography of the related art does not accurately measure exhaled carbon dioxide in certain situations. The explanation starts with an explanation of a capnographic waveform.

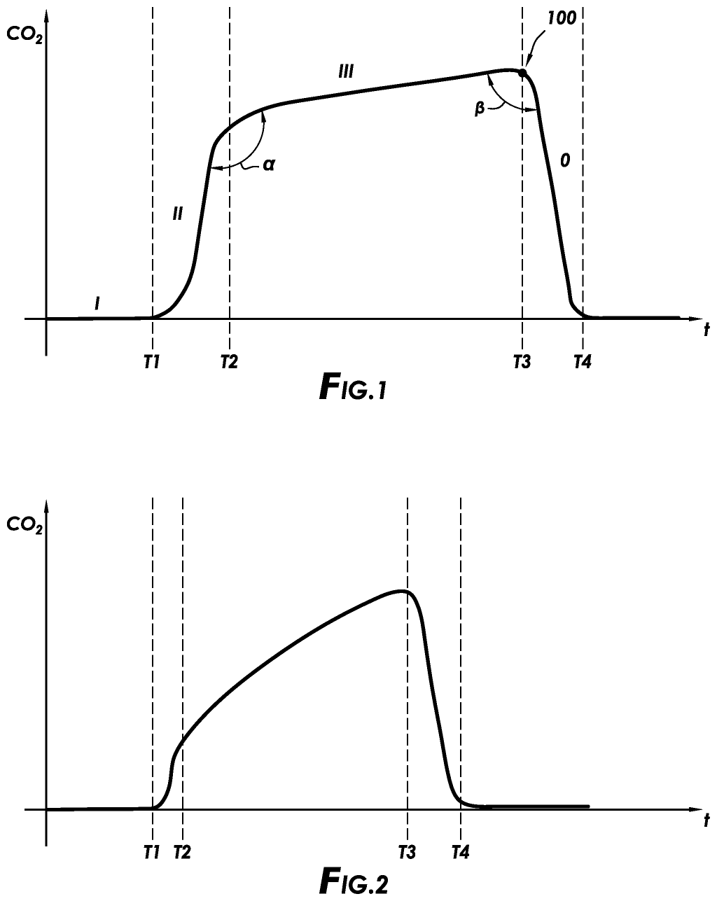

FIG. 1 shows a plot of a capnographic waveform in order to describe the various phases of exhalation. In particular, the example capnographic waveform of FIG. 1 is a plot of carbon dioxide in an exhalation as a function of time. The time period between the origin and a time T1 represents a period of time when the patient is inhaling, and is referred to in capnographic analysis as Phase I. To the extent the carbon dioxide of the inhaled gas is measured during the inhalation, the carbon dioxide measured during Phase I is the baseline carbon dioxide in ambient air, a relatively low value from a metabolic standpoint. The time period between time T1 and time T2 is the beginning of an exhalation, and is referred to in capnographic analysis as Phase II. The time period between time T2 and time T3 is the exhalation, and is referred to in capnographic analysis as Phase III or the alveolar plateau. The carbon dioxide in the exhalation at the end of Phase III, shown as point 100, is referred to as the end-tidal carbon dioxide (hereafter end-tidal carbon dioxide 100). In the presence of normally functioning lungs, the end-tidal carbon dioxide 100 is closely related to the amount of carbon dioxide within the patient's bloodstream. In some cases, the end-tidal carbon dioxide 100 can be taken directly to be the amount of carbon dioxide in the patient's bloodstream. The time period between time T3 and time T4 is the inspiratory down stroke at the beginning of an inhalation, and is referred in capnographic analysis as Phase 0.

The capnographic waveform of FIG. 1 is an ideal case for a normally functioning human. A capnographic waveform deviating from the ideal case of FIG. 1 may provide information regarding the underlying state of the patient. For example, the angle α between Phase II and Phase III is an indication of the ventilation/perfusion (V/Q) ratio of the exhalation. The angle β between Phase III and Phase 0 is an indication rebreathing. That is, if rebreathing is taking place, the angle β is greater than 90 angular degrees.

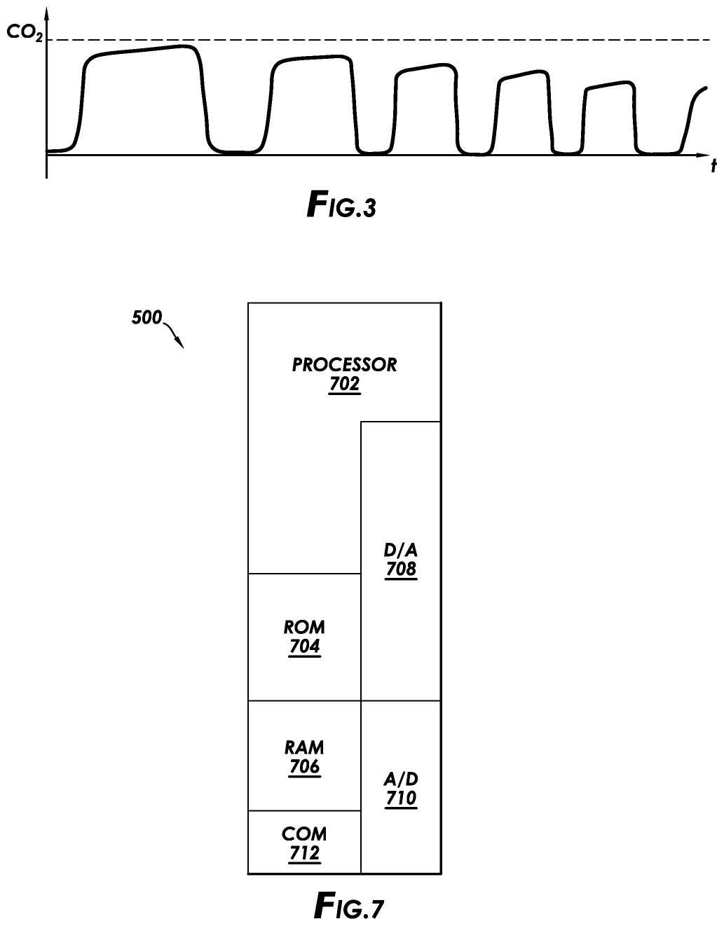

FIG. 2 shows a plot of another example capnographic waveform. In particular, the example capnographic waveform of FIG. 2 is a plot of carbon dioxide in an exhalation as a function of time. The example waveform of FIG. 2 takes a “shark fin” shape, with decreased Phase II rise time, and a sloped alveolar plateau in Phase III. The shark fin shaped capnographic waveform is associated with the underlying presence of asthma, bronchospasm, chronic obstructive pulmonary disease (COPD), and/or an obstruction or foreign body in the lungs (e.g., a mucus plug).

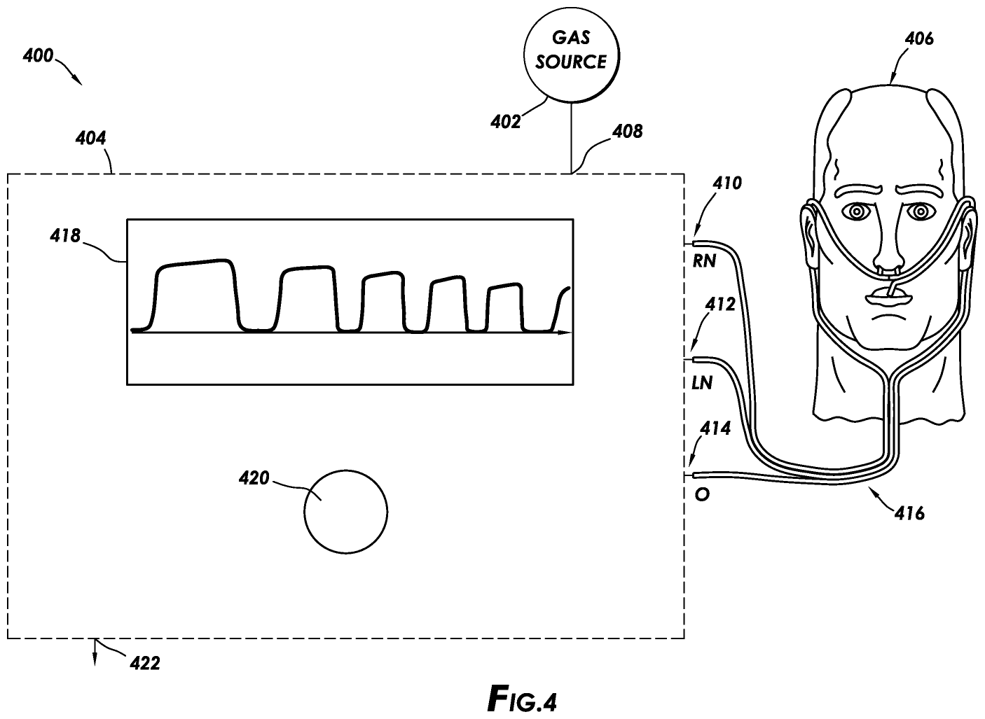

While a single capnographic waveform may provide valuable information to a clinician, a series of capnographic waveforms representing a plurality of exhalations may provide yet still further information. FIG. 3 shows a series of capnographic waveforms. In particular, the example capnographic waveforms of FIG. 3 are a plot of carbon dioxide in exhalations as a function of time. FIG. 3 shows five full and one partial capnographic waveform. Each successive exhalation has a lower end-tidal carbon dioxide, as shown by the successively lower transitions between Phase III and Phase 0. The situation depicted in FIG. 3 represents a hyperventilation of the underlying patient. That is, during the hyperventilation the carbon dioxide within the patient's bloodstream is lowered with each breath, and correspondingly the end-tidal carbon dioxide of each capnographic waveform is lower with each breath.

The example capnographic waveforms of FIGS. 1, 2, and 3, and many more, were developed in the context of a single-lumen exhalation path to a capnographic measurement system. More particularly, the capnographic waveforms used in the industry were developed in the context of a patient with a tracheal intubation such that all the inhaled gas and all the exhaled gas moves through a single lumen of an intubation tube. The intubation tube obviates any effects of the epiglottis and/or pharynx, and bypasses entirely the nasopharynx. Nevertheless, the industry may still rely on the example waveforms when other methods and systems are used to capture a portion of the exhaled gas for measurement. For example, in some cases a mask may be placed over the patient's nose and mouth. Exhaled gas from the lungs further interacts with the epiglottis and pharynx, and assuming the patient's mouth is closed, the exhaled gas also interacts with the nasopharynx, before entering the common-plenum of the mask.

In yet still other cases, a single-lumen nasal cannula may be used. That is, the single tube of the single-lumen nasal cannula wraps behind the patient's ears and under the patient's nose. The tube has two prongs or ports, with a left port in operational relationship to the left naris and a right prong port in operational relationship to the right naris. When the patient exhales, the exhaled gas from the lungs interacts with the epiglottis and pharynx, and also the nasopharynx, before encountering the ports of the single-lumen nasal cannula. A portion of the exhaled gas from the left naris enters the left prong, and a portion of the exhaled gas from the right naris enters the right prong. Exhaled gas that enters the single tube through the ports splits and flows along the tube in opposite directions, over the patient's ears. The two ends of the tube join together into a single lumen connector, and couple to the capnographic device. The capnographic device, in turn, measures carbon dioxide in the exhaled gas and provides an indication of the carbon dioxide (e.g., to a clinician). The indication of carbon dioxide may take any suitable form, such as percentage of carbon dioxide relative to other exhaled gasses, volume of carbon dioxide in the exhaled gasses, mole percentage of carbon dioxide in the exhaled gasses, and/or partial pressure of the carbon dioxide in the exhaled gasses. Hereafter, the indication is referred to as “carbon dioxide percentage” with the understanding that the term contemplates any suitable measure of the relationship of carbon dioxide to the remaining gases in the exhaled gas.

For purposes of highlighting the shortcomings of related art methods, consider a situation where exhaled gas flows evenly out of each naris (e.g., 50% left naris, 50% right naris, and mouth closed). In this situation, the pressure of exhaled gas entering the left port and right port would be equal, and thus the flow of exhaled gas into each port would be equal. However, it is rare that exhaled gas is evenly distributed across the left naris and right naris. The nasal passages and other nasal features may not be mirror images, and one nasal passage may have larger or smaller cross-sectional flow area, leading to different flow rates out of each naris. It follows that, even when each nasal passage is open to its fullest extent, the exhaled gas flow as between the nares may be unbalanced or different. Moreover, humans experience what is known as the “nasal cycle.” In nasal cycle the nasal passage most open to flow shifts back and forth throughout the course of the day or night. For example, initially the left nasal passage may be open and the right nasal passage may be fully blocked. Later, the right nasal passage may begin to open to flow such that flow is divided (not necessarily evenly). Later still, the left nasal may be fully blocked and only the right nasal may be open to flow.

The inventor of the current specification has found that dynamic changes to exhaled gas flow, such as created by the nasal cycle, adversely affect the ability of related art capnographic devices to accurately measure carbon dioxide percentage. Referring again to the situation in which a single-lumen nasal cannula is used, and with one nasal passage (e.g., left nasal passage) fully blocked and the mouth closed. During an exhalation, the exhaled gas flows solely out the right naris, and a portion of the exhaled gas enters the right port of the single-lumen nasal cannula. However, because the left nasal passage is fully blocked in this example, the left port is effectively at atmospheric pressure. Because the right port is at pressure higher than atmospheric, the exhaled gas entering the right port at higher than atmospheric pressure has two possible paths: 1) through the length of the tubing, into the capnographic device, past the carbon dioxide sensor, and then to atmosphere; or 2) through the single lumen from the right port to the left port. The path from the right port to the left port is a significantly shorter path and thus has lower resistance to gas flow. It follows that a portion, in fact a substantial portion, of the exhaled gas that enters the right port “leaks” back out the left port rather than flowing to the sensor in the measurement device. Of course, when the left nasal passage is open and the right nasal passage is fully blocked, a similar “leak” occurs from the left port to the right port. The “leak” may also occur in situations where one nasal passage is only slightly open to flow, and thus cannot develop sufficient pressure at its respective port to overcome the pressure of the “leaking” exhaled gas. An understanding of why a change in the flow of exhaled gas may cause a change in carbon dioxide percentage read by the sensor in the capnographic device becomes apparent after a discussion of sensors used to measure carbon dioxide percentage.

Sensors for measuring carbon dioxide percentage require a finite measurement period or duration. For example, spectroscopic sensors transmit ultraviolet light through a sample, and then measure absorption of the ultraviolet light as an indication of the carbon dioxide percentage. While light travels very fast, photo sensors require a finite amount of time to capture sufficient photons to derive a measurement value. Similarly from a measurement duration standpoint, chemical carbon dioxide sensors derive a carbon dioxide percentage by sensitivity of polymer- or heteropolysiloxane layers, and these devices do not and cannot respond instantaneously to changes in carbon dioxide percentage. In both cases, the flow of exhaled gas may control how quickly the sensor settles on the actual carbon dioxide percentage, with lower flow adversely affecting the measured carbon dioxide percentage if measurement duration is held constant. It is to be understood that the flow rate of exhaled gas does not change the actual carbon dioxide percentage in the exhaled gas; rather, lower than designed or expected flow rates in combination with measurements that require a finite measurement duration may result in readings that are not accurate when flow of exhaled gas is low.

Returning to FIGS. 1 and 2. The capnographic waveforms of FIGS. 1 and 2 assume tracheal intubation of the patient and responsiveness of the underlying carbon dioxide sensor. However, leaks of exhaled gas significantly reduce the volume of exhaled gas applied to the carbon dioxide sensor, and thus the leaks may affect accuracy and response time of the carbon dioxide measurement. In fact, the changes in the capnographic waveforms as between FIGS. 1 and 2 may be attributable to leaks of the exhaled gas as discussed above. Consider that the capnographic waveform of FIG. 1 is created during a period of time when both nasal passages are open to flow and thus equal amounts of exhaled gas enter the ports of the single-lumen nasal cannula. In such a situation, sufficient flow of exhalation gas is forced along the tube of the single-lumen nasal cannula to the carbon dioxide sensor. Now consider that one nasal passage becomes clogged or blocked, and thus the exhaled gas is provide to only one port of the single-lumen nasal cannula. As discussed above, because of the leak of exhaled gas through the unused port, the volume of exhaled gas applied to the carbon dioxide sensor may drop significantly— not just by half, but in many cases by 75% or more. The lowered volume of exhaled gas applied to the carbon dioxide sensor may adversely affect responsiveness of the sensor and/or the accuracy, thus lowering the measured carbon dioxide percentage. It follows that the capnographic waveform of FIG. 2 may represent something as benign as one nasal passage of the patient becoming clogged or blocked. Nevertheless, if not taken into account, the change may be considered to represent a serious internal issue (e.g., lung blockage, mucus plug) rather than just a normal change associated with the nasal cycle.

Now consider again FIG. 3. The first capnographic waveform may represent the carbon dioxide measurement as a function of time when both nasal passages are open to flow. Now consider that, over the course of several breaths, one nasal passage of the patient becomes progressively more clogged or blocked. As the flow of exhalation gas from one nasal passages trends downward, more and more exhaled gas from the open nasal passage “leaks” through the associated port. The lowered volume of exhaled gas to the carbon dioxide sensor in each exhalation may manifest itself as a lower measured carbon dioxide percentage in each exhalation. It follows that the series of capnographic waveforms of FIG. 3 may represent something as benign as one nasal passage of the patient becoming clogged or blocked. Nevertheless, if not taken into account, the change may be considered to represent hyperventilation rather than just a normal change associated with nasal cycle.

A similar issue may exist, though possibly to a lesser degree, in the case of a nasal mask covering the patient's nose and mouth. One nasal passage becoming clogged due to nasal cycle may manifest itself as the shark fin case of the waveform of FIG. 2, which again may lead to misdiagnosis. That is to say, a blockage of one nasal passage because of the nasal cycle, if not taken into account, may be considered to represent a serious internal issue (e.g., lung blockage, mucus plug) rather than just a normal change associated with the nasal cycle.

Various example embodiments address, in whole or in part, the issues noted above by separately sensing exhaled gas from each breathing orifice (e.g., left naris, right naris, and mouth). In one example case, a single breathing orifice with sufficient exhaled gas flow is selected, and measuring carbon dioxide percentage is performed only with respect to the selected breathing orifice. In cases where three breathing orifices exhibit sufficient flow, the measurement of carbon dioxide percentage from all three breathing orifices may be combined in any suitable form, such as the square root transform. If only two of the three breathing orifices exhibit sufficient flow (e.g., mouth closed but both nasal passages open), then only the measurement of carbon dioxide percentage associated with the nares may be used and combined in any suitable way, such as by averaging the respective carbon dioxide percentages. If only one breathing orifice exhibits sufficient flow (e.g., just the left naris), then only the carbon dioxide readings associated with the left naris is used and provided to the clinician.

While in some cases capnography is performed with a standalone device, in example embodiments the capnography is performed in conjunction with oxygen delivery by way of an oxygen delivery system. An oxygen delivery system may sense a patient's inhalation or inspiration, and deliver a bolus of therapeutic gas only during inspiration. Delivering only during inhalation reduces the use of therapeutic gas, and thus such oxygen delivery systems are sometimes referred to as conservers. The inventor of the present specification is also a co-inventor of U.S. Pat. No. 7,007,692 titled “Method and system of sensing airflow and delivering therapeutic gas to a patient” (hereafter the '692 patent). The '692 patent contemplates individually sensing airflow of the breathing orifices (e.g., by a pressure sensor, or a flow sensor) and preferentially delivering therapeutic gas to the breathing orifice or orifices that have airflow. In this way, the '692 patent may teach reducing waste of therapeutic gas by not attempting to deliver to a breathing orifice that cannot accept the therapeutic gas. In addition to the oxygen delivery during inhalation, example oxygen delivery systems also generate capnographic waveforms during exhalation, similar to the capnographic waveforms of FIGS. 1-3. The specification now turns to a description of various embodiments which address, at least in part, the shortcomings noted in the context of also operating as a conserver used to provide therapeutic gas.

FIG. 4 shows a system 400 in accordance with at least some embodiments. The example system 400 comprises a gas source 402, a combined conserver and capnography device 404 (hereafter device 404), and a patient 406. The device 404 defines a gas port 408, a right naris (RN) hose connection 410, a left naris (LN) hose connection 412, and oral (0) hose connection 414. The gas source 402 is coupled to the gas port 408, and the gas source 402 may take any suitable form. For example, the gas source 402 may be an oxygen concentrator system, a gas cylinder, or a permanent supply system, such as in a hospital. The example device 404 couples to the patient 406 by way of the hose connections and a nasal cannula 416. In the example system 400 of FIG. 4, the nasal cannula 416 is a three-lumen nasal cannula. That is, the nasal cannula 416 has a first tube or first lumen that fluidly couples between the right naris hose connection 410 and the right naris of the patient 406. The nasal cannula 416 has a second tube or second lumen that fluidly couples between the left naris hose connection 412 and the left naris of the patient 406, and the second lumen is fluidly isolated from the first lumen. The nasal cannula 416 has third tube or third lumen that fluidly couples between the oral hose connection 414 and the mouth of the patient 406, and the third lumen is fluidly isolated from the first and second lumens. While a three-lumen nasal cannula is shown, in other cases the operations with respect to the mouth may be omitted, and in such cases the nasal cannula 416 may be a dual-lumen or bifurcated nasal cannula defining two fluidly isolated lumens.

The example device 404 further comprises a display 418 on which information may be presented to the user. In the example shown, the display 418 shows a series of capnographic waveforms, such as may be created by the device 404 monitoring exhalations and reading carbon dioxide percentage of exhaled gas in each exhalation. In addition to, or in place of, the display 418, the capnographic waveforms may be sent to external devices, such as by data over a data communication port 422. The example device 404 may further comprises a control knob 420 with which the user, such as the patient 406, interacts with the device 404.

In accordance with at least some embodiments, the device 404 monitors inhalations of the patient 406 and delivers a bolus of therapeutic gas at the beginning of each inhalation. In particular, the device 404 delivers to the left naris, the right naris, or to the mouth of the patient, when one or more of those locations are open to flow. More particularly, in cases where only one breathing orifice is open to flow, the device 404 delivers to only one breathing orifice. In cases where two or more breathing orifices are open to flow, the example device 404 may choose a delivery location, deliver to all, or alternate the delivery location breath to breath.

During each exhalation, the example device 404 measures carbon dioxide in the exhaled gas flow through each breathing orifice, and creates a set of values indicative of carbon dioxide (e.g., carbon dioxide percentage) one each for each breathing orifice. The example device 404 creates a capnographic waveform using one set of values, two sets of values, or all three sets of values depending on the circumstances of the patient 406. Consider a first case in which only one nasal passage is open to flow of exhaled gas, the second nasal passage is clogged or blocked, and the mouth is closed. In this first case the capnographic waveform for each exhalation may directly be the set of values indicative of carbon dioxide measured for the nasal passage open to flow. Each value of the set of values may represent a carbon dioxide percentage at a particular point in time of the exhalation, or alternatively each value of the set of values may represent a carbon dioxide percentage as a particular accumulated volume of the exhalation.

Now consider a second case in which in which both nasal passages are open to flow of exhaled gas, and the mouth is closed. During an exhalation, exhaled gas may thus flow through the right naris and the left naris. The example device 404 measures carbon dioxide percentage of the exhaled gas through the right nasal passage (e.g., by way of the right naris hose connection 410), and creates a first set of values indicative of carbon dioxide. Simultaneously, the example device 404 measures carbon dioxide percentage of the exhaled gas through the left nasal passage (e.g., by way of the left naris hose connection 412), and creates a second set of values indicative of carbon dioxide. If the flow of exhaled gas through either the flow path associated with the right naris hose connection 410 or the flow path associated with the left naris hose connection 412 is below a predetermined threshold, then the set of values created associated with the nasal passage may be ignored or omitted for purposes of creating the capnographic waveform for the exhalation. For example, if the flow of exhaled gas is below a threshold flow of the carbon dioxide sensor for the flow path, the carbon dioxide percentage created by the carbon dioxide sensor may be suspect, and the set of values may be ignored or omitted when creating the capnographic waveform for the particular exhalation. In such a situation, the set of values indicative of carbon dioxide associate with the flow path whose flow is above the predetermined threshold may be directly assigned to be the capnographic waveform for the exhalation.

Still considering the second case in which both nasal passages are open to flow of exhaled gas and the mouth is closed, now consider that the flow of exhaled gas in each flow path is above the predetermined threshold. In example cases the device 404 creates the capnographic waveform for the exhalation by selecting one set of values indicative of carbon dioxide to directly be the capnographic waveform. In other cases, the device 404 may create the capnographic waveform by combining the set of values indicative of carbon dioxide associated with the flow path of the right naris hose connection 410 with the set of values indicative of carbon dioxide associated with the flow path of the left naris hose connection 412. If the set of values indicative of the carbon dioxide associated with the right naris are assigned the mathematical form RN(X), and the set of values indicative of the carbon dioxide associated with the left naris are assigned the mathematical form LN(X) (e.g., “X” may be time within the exhalation, or “X” may be accumulated volume of the exhalation), then creating the capnographic waveform may comprise computing or calculating a combined value for each corresponding value of “X”. In one example case, calculating the combined value at each corresponding value of “X” may involve averaging the values in the sets of values indicative of carbon dioxide. If “X” is time, then the example method is averaging the corresponding values of the first and second sets of values indicative of carbon dioxide at corresponding points in time. If “X” is accumulated volume of the exhalation, then the example method is averaging the values of the first and second sets of values indicative of carbon dioxide at corresponding points in accumulated volume.

Now consider a third case in which in which both nasal passages are open to flow of exhaled gas, and the mouth is open to flow of exhaled gas. During an exhalation, exhaled gas may thus flow through the right naris, the left naris, and the mouth. The example device 404 simultaneously: measures carbon dioxide percentage of the exhaled gas through the right nasal passage (e.g., by way of the right naris hose connection 410), and creates a first set of values indicative of carbon dioxide; measures carbon dioxide percentage of the exhaled gas through the left nasal passage (e.g., by way of the left naris hose connection 412), and creates a second set of values indicative of carbon dioxide; and measures carbon dioxide percentage of the exhaled gas through the mouth (e.g., by way of the oral hose connection 414), and creates a third set of values indicative of carbon dioxide. If the flow of exhaled gas through any particular flow path is below the predetermined threshold, then the set of values created associated with the flow path may be ignored or omitted for purposes of creating the capnographic waveform for the exhalation. If only one flow path has a flow of exhaled gas above the threshold, then the set of values indicative of carbon dioxide associated with the flow path whose flow is above the predetermined threshold may be directly assigned to be the capnographic waveform for the exhalation.

In the third case being considered, if the flow of exhaled gas is above the threshold for two of the three flow paths, then the one of the sets of values indicative of carbon dioxide associated with the flow paths whose flow is above the predetermined may be selected to directly be the capnographic waveform. In other cases, the device 404 may create the capnographic waveform by combining the two sets of values indicative of carbon dioxide associated with the flow paths whose flow is above the predetermined threshold. For example, the device 404 may average the corresponding values in the sets of values indicative of carbon dioxide.

Still in the third case being considered, if the flow of exhaled gas is above the threshold for all three flow paths, then again the one of the sets of values indicative of carbon dioxide associated with the flow paths whose flow is above the predetermined may be selected to directly be the capnographic waveform. In other cases, the device 404 may create the capnographic waveform by combining the three sets of values indicative of carbon dioxide. For example, the device 404 may average the corresponding values in the three sets of values indicative of carbon dioxide. The specification now turns to an example device 404 in greater detail.

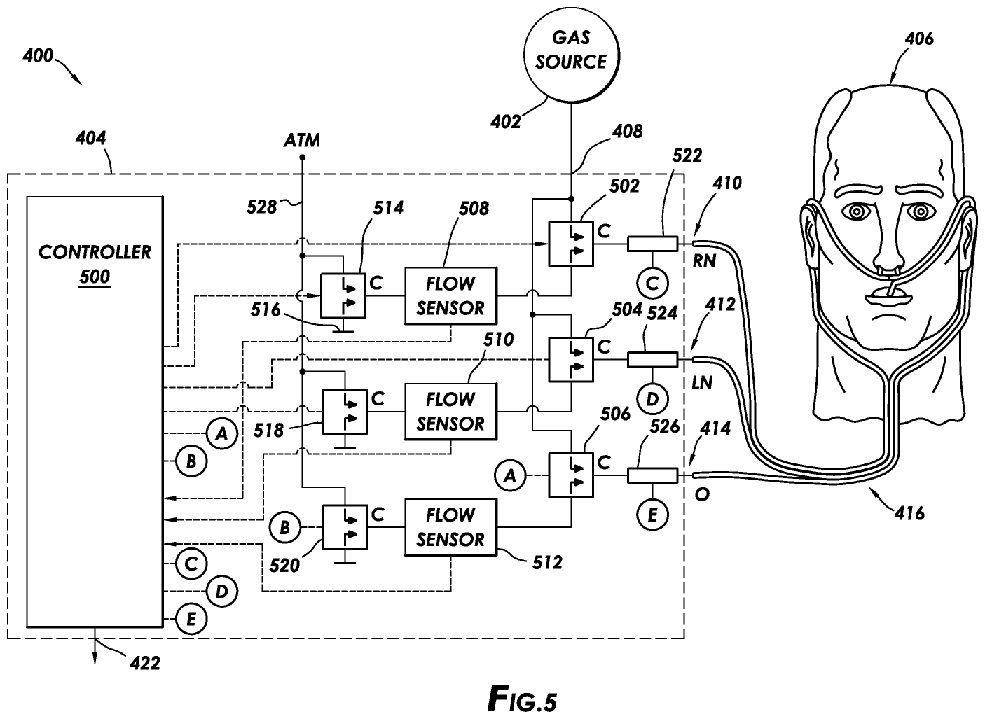

FIG. 5 shows an example system. In particular, the example system 400 comprises the device 404, the patient 406, and the three-lumen nasal cannula 416. FIG. 5 further shows, in block diagram form, and example device 404. The device 404 comprises both electrical components and mechanical connections. In order to differentiate between electrical connections and mechanical connections, FIG. 5 illustrates electrical connections between components with dashed lines, and fluid connections (e.g., tubing connections between devices) with solid lines. The example device 404 of FIG. 5 comprises controller 500. The example controller 500 may drive on/off or Boolean signals, such as signals to control the state of the various electrically-controlled valves. The example controller 500 reads signals (e.g., analog signals from various sensors) indicative inhalations through the breathing orifices as well as indicative of carbon dioxide percentage in exhalations, and creates capnographic waveforms as discussed above. In some cases, the capnographic waveforms are sent to other devices over the data communications port 422 (e.g., serial communications). In some cases the controller 500 may show one or more capnographic waveforms on the display 418 (not shown in FIG. 5 so as not to further complicate the figure).

The example device 404 comprises electrically-controlled valves in the form of three-port valve 502, three-port valve 504, and three-port valve 506. In accordance with various embodiments, each of these three-port valves may be a five-volt solenoid operated valve that selectively couples one of two ports to a common port (each common port labeled as C in the figure). Three-port valves 502, 504, and 506 may be Humprey Mini-Mizers having part No. D3061, such as may be available from the John Henry Foster Co., or equivalents. Each three-port valve 502, 504, and 506 is electrically coupled to the controller 500. By selectively applying voltage from the controller 500 on a respective electrical connection, the controller 500 may be able to control the state of the device 404. For example, with respect to the three-port valve 502, the three-port valve 502 may: couple gas from the gas port 408 to the common port and therefore to the example right naris hose connection 410; and couple a sensor in the example form of flow sensor 508 to the common port and therefore the example right naris hose connection 410. Likewise, the three-port valve 504, under command of the controller 500, may: couple gas from the gas port 408 to the common port and therefore the example left naris hose connection 412; and couple a sensor in the example form of a flow sensor 510 to the common port and therefore the example left naris hose connection 412. Further still, three-port valve 506 under command from the controller 500 may: couple gas from the gas port 408 to the common port and to the oral hose connection 414; and couple a sensor in the example form of flow sensor 512 to the common port and to the oral hose connection 414.

The example flow sensors 508, 510, and 512 are electrically coupled to the controller 500 such that the controller 500 can read the flow sensed by each. More particularly, the controller 500 may read values indicative of airflow (e.g., inhalation by the patient) through each respective breathing orifice. In alternative embodiments, the flow sensors 508, 510, and 512 may couple to the common ports of the three-port valves 502, 504, and 506, respectively, if the flow sensors can withstand the pressure of the therapeutic gas during bolus delivery without damage. In yet still further embodiments, pressure sensors may be used in place of the flow sensors, the pressure sensors placed at any suitable location (e.g., the common ports of each three-port valve 502, 504, and 506). Regardless of the precise type and placement of the sensors, the controller 500 may be able to determine when the patient is inhaling, and in some cases an indication of how much of the air drawn by the patient flows through each of the monitored breathing orifices. Moreover, the controller 500 may be able to determine when the patient is exhaling, and in some cases an indication of how much of the exhaled gas is carried by each breathing orifice.

In the example system 400 of FIG. 5, each of the flow sensors is designed and constructed to enable gas flow through the sensor, hence the reason for the terminology “flow sensor.” Because the example system 400 measures inhalation airflow and provides a bolus of therapeutic gas during each inhalation, the use of flow sensors creates the possibility that therapeutic gas flow from the gas source 402 can “leak” through the device 404 and not reach the patient 406. That is, and considering flow sensor 508 as representative, during a period of time when the device 404 provides therapeutic gas to the right naris, the three-port valve 502 provides the therapeutic gas to the right naris and blocks flow through the flow sensor 508. After the bolus delivery ends, the three-port valve 502 changes valve position and thus fluidly couples the flow sensor 508 to the common port and therefore the example right naris. If the flow sensor 508 outlet is not blocked for a further portion of the inhalation, a portion of the therapeutic gas may reverse flow through the flow sensor 324. Thus, in the example device 404 an electrically-controlled valve in the form of three-port valve 514 couples between the flow sensor 508 and the atmospheric vent (labeled ATM in the figure).

Three-port valve 514, in a first valve position, couples the flow sensor 508 to the atmospheric vent ATM, thus enabling gas flow (e.g., inhalation gas flow, flow of exhaled gas) through the flow sensor 508 for measurement purposes. The three-port valve 514, in a second valve position, couples the flow sensor 508 to a blocked port 516 to prevent the reverse flow of therapeutic gas. Thus, three-port valve 514 (as well as corresponding three-port valves 518 and 520) may be used to temporarily block reverse flow and loss of therapeutic gas. In some cases, the three-port valves 514, 518, and 520 may remain in a position that blocks flow for about 300 milliseconds after therapeutic gas delivery has stopped by a upstream three-port valves 502, 504, and 506. After the expiration of the period of time of possible reverse flow has ended, the three-port valves 514, 518, and 520 change valve positions, thus enabling the flow sensors to again sense airflow (e.g., during an upcoming exhalation).

Still referring to FIG. 5, the example device 404 further comprises carbon dioxide sensors associated with each flow path. In particular, the example device 404 comprises a carbon dioxide sensor 522 fluidly coupled between the three-port valve 502 and the right naris hose connection 410. The carbon dioxide sensor 522 is electrically coupled to the controller 500. The carbon dioxide sensor 522 measures carbon dioxide in the flow of exhaled gas that moves through the flow path associated with the right naris hose connection 410, and provides values indicative of carbon dioxide to the controller 500. The example device 404 further comprises a carbon dioxide sensor 524 fluidly coupled between the three-port valve 504 and the left naris hose connection 412. The carbon dioxide sensor 524 is electrically coupled to the controller 500. The carbon dioxide sensor 524 measures carbon dioxide in the flow of exhaled gas that moves through the left naris hose connection 412, and provides values indicative of carbon dioxide to the controller 500. The example device 404 further comprises a carbon dioxide sensor 526 fluidly coupled between the three-port valve 506 and the oral hose connection 414. The carbon dioxide sensor 526 is electrically coupled to the controller 500. The carbon dioxide sensor 526 measures carbon dioxide in the flow of exhaled gas that moves through the oral hose connection 414, and provides values indicative of carbon dioxide to the controller 500.

Using the carbon dioxide sensors 522, 524, and 526, the device 404 may create sets of values indicative of carbon dioxide during respiration. As discussed above, the controller 500 may then select one of the sets of values of carbon dioxide to be the capnographic waveform for the exhalation, or the controller may combine two or more sets of values of carbon dioxide to create the capnographic waveform for the exhalation.

The carbon dioxide sensors 522, 524, and 526 may take any suitable form. For example, the carbon dioxide sensors 522, 524, and 526 may be ultraviolet-type sensors in which the carbon dioxide percentages are derived from how much ultraviolet light is absorbed by the carbon dioxide in the exhaled gas. In other cases, the carbon dioxide sensors 522, 524, and 526 may be membrane-type sensors in which the carbon dioxide percentages are derived from how much gas permeates the membrane of each sensor.

FIG. 5 shows the example carbon dioxide sensors 522, 524, and 526 disposed between their respective three-port valves and their respective hose connections. However, the carbon dioxide sensors 522, 524, and 526 may be placed at any suitable location at which the flow of exhaled gases may be measured. For example, each carbon dioxide sensor may be fluidly coupled between the three-port valves of a flow path. Considering the flow path associated with the right naris hose connection 410 as an example, the carbon dioxide sensor 522 may be fluidly coupled between the three-port valve 502 and the three-port valve 514 on either side of the flow sensor 508. In yet still other cases, the carbon dioxide sensors may be placed downstream of the blocking three-port valves 514, 518, and 520. Considering again the flow path associated with the right naris hose connection 410 as an example, the carbon dioxide sensor 522 may be fluidly coupled between the three-port valve 514 and a header 528 for the atmospheric vent.

In some cases, the conserver action of the device 404 may be conceptually separated from the capnography aspects. That is, the device 404 may provide a bolus of therapeutic gas based on the prescription titration flow rate at the beginning of each inhalation regardless of end-tidal carbon dioxide percentage. However, in yet still other cases the device 404, and particularly the controller 500, may change or adjust the bolus size (for bolus delivery systems) or change or adjust the flow rate (for interrupted continuous flow systems that flow at a titration prescription flow rate for a predetermined portion of each inhalation) based on the end-tidal carbon dioxide percentage. In particular, high end-tidal carbon dioxide percentage may be indicative of higher metabolic rate in acute patients (e.g., with reduced lung function), and thus indicative of too little oxygen being delivered. Oppositely, lower end-tidal carbon dioxide percentage may be indicative lower metabolic rate in acute patients, and thus indicative of too much oxygen being delivered. Thus, in example embodiments the device 404 increases bolus size (e.g., increases on the next inhalation) when end-tidal carbon dioxide percentage is above a first predetermined threshold. Alternately, the device 404 decreases bolus size (e.g., decreases on the next inhalation) when end-tidal carbon dioxide percentage is below a second predetermined threshold. There may be an operating window between the first and second predetermined thresholds in which no change to bolus size and/or flow is made.

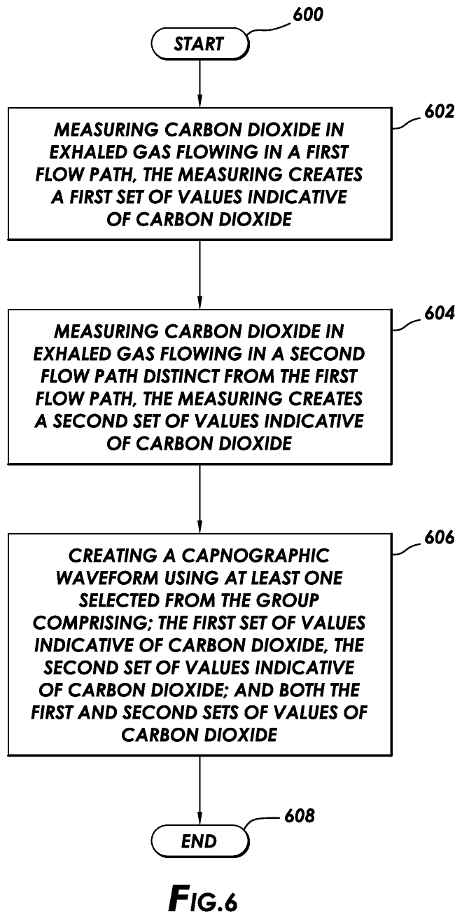

FIG. 6 show a method in accordance with at least some embodiments. In at least some cases, the example method may be performed by the controller 500 (e.g., instructions executed by a processor). In particular, the method starts (block 600). The method may then comprise measuring carbon dioxide in exhaled gas flowing in a first flow path, the measuring creates a first set of values indicative of carbon dioxide (block 602). The method may comprise measuring carbon dioxide in exhaled gas flowing in a second flow path distinct from the first flow path, the measuring creates a second set of values indicative of carbon dioxide (block 604). And the method may then comprise creating a capnographic waveform using at least one selected from the group comprising; the first set of values indicative of carbon dioxide; the second set of values indicative of carbon dioxide; and both the first and second sets of values of carbon dioxide (block 606). Thereafter, the method ends (block 608), likely to be restarted on the next exhalation.

FIG. 7 shows a controller 500 in accordance with at least some embodiments. The example controller 500 may be microcontroller, and therefore the microcontroller may be integral a processor 702, read only memory (ROM) 704, random access memory (RAM) 706, a digital-to-analog converter (D/A) 708, and an analog-to-digital converter (ND) 710. The controller 500 may further comprise communication logic 712, which enables systems to communicate with external devices, e.g., to transfer capnographic waveforms to external devices. Although a microcontroller may be preferred because of the integrated components, in alternative embodiments the controller 500 may be implemented by a stand-alone processor 702 in combination with individual RAM, ROM, communication, D/A and ND devices, in addition to, or in place of, any of the further components and devices noted the Definitions section above.

The ROM 704 may store instructions executable by the processor 702. In particular, the ROM 704 may comprise a software program or instructions that, in whole or in part, implements the various embodiments discussed herein. The RAM 706 may be the working memory for the processor 702, where data may be temporarily stored and from which instructions may be executed. Processor 702 may couple to other devices within the delivery system by way of A/D converter 710 (e.g., sensors to sense attributes of airflow, carbon dioxide sensors) and D/A converter 708 (e.g., electrically-controlled valves). Thus, the ROM 704, and/or the RAM 706 may be non-transitory computer-readable mediums upon which instructions are stored.

The above discussion is meant to be illustrative of the principles and various embodiments of the present invention. Numerous variations and modifications will become apparent to those skilled in the art once the above disclosure is fully appreciated. For example, a single carbon dioxide sensor could be used at a common plenum location, such as just before the atmospheric vent. It is intended that the following claims be interpreted to embrace all such variations and modifications.

What is claimed is:

1. A method of generating a capnographic waveform, the method comprising:
measuring, by a controller of a device, carbon dioxide in exhaled gas flowing in a first flow path, the measuring creates a first set of values indicative of carbon dioxide;
measuring, by the controller of the device, carbon dioxide in exhaled gas flowing in a second flow path distinct from the first flow path, the measuring creates a second set of values indicative of carbon dioxide; and
creating, by the controller of the device, a capnographic waveform using at least one selected from the group comprising: the first set of values indicative of carbon dioxide; the second set of values indicative of carbon dioxide; and
both the first and second sets of values indicative of carbon dioxide.

2. The method of claim 1:
wherein measuring exhaled gas flow in the first flow path further comprises measuring exhaled gas flow from a first lumen of a nasal cannula; and
wherein measuring exhaled gas flow in the second flow path further comprises measuring exhaled gas flow from a second lumen of the nasal cannula, the first lumen fluidly isolated from the second lumen.

3. The method of claim 1:
wherein measuring exhaled gas flow in the first flow path further comprises measuring exhaled gas flow from a first lumen of a bifurcated nasal cannula; and
wherein measuring exhaled gas flow in the second flow path further comprises measuring exhaled gas flow from a second lumen of the bifurcated nasal cannula.

4. The method of claim 1 wherein creating the capnographic waveform further comprises combining the first and second sets of values indicative of carbon dioxide.

5. The method of claim 4 wherein combining the first and second sets of values indicative of carbon dioxide further comprises averaging corresponding values in the first and second sets of values indicative of carbon dioxide.

6. The method of claim 5 wherein averaging corresponding values further comprises averaging the values of the first and second sets of values indicative of carbon dioxide at corresponding points in time.

7. The method of claim 5 wherein averaging corresponding values further comprises averaging the values of the first and second sets of values indicative of carbon dioxide at corresponding points in accumulated volume of exhaled gas.

8. The method of claim 1 further comprising:
sensing an inhalation; and
providing a volume of therapeutic gas triggered by the sensing and based on the capnographic waveform.

9. A system comprising:
a controller;
a first carbon dioxide sensor (CO₂ sensor) fluidly coupled to a first hose connection, and communicatively coupled to the controller;
a second CO₂ sensor fluidly coupled to a second hose connection, and communicatively coupled to the controller;
wherein the controller is configured to:
read a first set of values indicative of carbon dioxide from the first CO₂ sensor;
read a second set of values indicative of carbon dioxide from the second CO₂ sensor;
create a capnographic waveform using at least one of the first and second sets of values indicative of carbon dioxide.

10. The system of claim 9 further comprising:
a first sensor electrically coupled to the controller and configured to fluidly couple to the first hose connection, the first sensor senses an attribute of airflow through the first hose connection;
a second sensor electrically coupled to the controller and configured to fluidly couple to the second hose connection, the second sensor senses an attribute of airflow of the second hose connection;
wherein when the controller creates the capnographic waveform, the controller is further configured to:
read the attribute of airflow from the first sensor;
read the attribute of airflow from the second sensor;
utilize the first set of values indicative of carbon dioxide if the attribute of airflow from the first sensor is above a predetermined threshold; and
utilize the second set of values indicative of carbon dioxide if the attribute of airflow from the second sensor is above a predetermined threshold.

11. The system of claim 9 wherein when the controller creates the capnographic waveform, the controller creates the capnographic waveform using both the first and second sets of values indicative of carbon dioxide.

12. The system of claim 11 wherein when the controller creates the capnographic waveform, the controller is further configured to average corresponding values in the first and second sets of values indicative of carbon dioxide.

13. The system of claim 12 wherein when the controller averages, the controller is further configured to average the values of the first and second sets of values indicative of carbon dioxide at corresponding points in time.

14. The system of claim 12 wherein when the controller averages, the controller is further configured to average the values of the first and second sets of values indicative of carbon dioxide at corresponding points in accumulated volume.

15. The system of claim 9 further comprising:
a first sensor electrically coupled to the controller and configured to fluidly couple to the first hose connection, the first sensor senses an attribute of airflow through the first hose connection;
a first valve electrically coupled to the controller and configured to fluidly couple a source hose connection to the first hose connection;
a second sensor electrically coupled to the controller and configured to fluidly couple to the second hose connection, the second sensor senses an attribute of airflow of the second hose connection;
a second valve electrically coupled to the controller and configured to fluidly couple the source hose connection to the second hose connection;
wherein the controller is further configured to:
sense an inhalation by way of the first sensor or the second sensor;
provide, based on the sensing, a flow of therapeutic gas to the first hose connection by way of the first valve.

16. The system of claim 15 wherein the controller is further configured to modify a volume of therapeutic gas provided in a subsequent inhalation based on the capnographic waveform.

17. A system comprising:
a source of therapeutic gas;
a bifurcated nasal cannula;
a therapeutic gas delivery device coupled to the source of therapeutic gas, and coupled to the bifurcated nasal cannula, the therapeutic gas delivery device comprising:
a controller;
a first carbon dioxide sensor (CO₂ sensor) fluidly coupled to a first lumen of the bifurcated nasal cannula, and communicatively coupled to the controller;
a second CO₂ sensor fluidly coupled to a second lumen of the bifurcated nasal cannula, and communicatively coupled to the controller;
a first sensor electrically coupled to the controller and configured to fluidly couple to the first lumen of the bifurcated nasal cannula, the first sensor senses an attribute of airflow through the first lumen;
a first valve electrically coupled to the controller and configured to fluidly couple the source of therapeutic gas to the first lumen;
a second sensor electrically coupled to the controller and configured to fluidly couple to the second lumen of the bifurcated nasal cannula, the second sensor senses an attribute of airflow of the second lumen;
a second valve electrically coupled to the controller and configured to fluidly couple the source of therapeutic gas to the second lumen;
wherein the controller is configured to:
sense an inhalation by way of the first sensor or the second sensor;
provide a flow of therapeutic gas to the first lumen of the bifurcated nasal cannula;
read a first set of values indicative of carbon dioxide from the first CO₂ sensor during an exhalation;
read a second set of values indicative of carbon dioxide from the second CO₂ sensor during the exhalation; and
create a capnographic waveform using at least one of the first and second sets of values indicative of carbon dioxide.

18. The system of claim 17 further comprising wherein when the controller creates the capnographic waveform, the controller is further configured to:
read the attribute of airflow from the first sensor;
read the attribute of airflow from the second sensor;
utilize the first set of values indicative of carbon dioxide if the attribute of airflow from the first sensor is above a predetermined threshold; and
utilize the second set of values indicative of carbon dioxide if the attribute of airflow from the second sensor is above a predetermined threshold.

19. The system of claim 18 wherein when the controller creates the capnographic waveform, the controller is further configured to average corresponding values in the first and second sets of values indicative of carbon dioxide.

20. The system of claim 17 wherein the controller is further configured to control a volume of therapeutic gas delivered based on a capnographic waveform associated with a prior exhalation.
